(19) United States
(12) Patent Application Publication
(10) Pub. No.: US 2024/0139359 A1
(43) Pub. Date: May 2, 2024

(54) Circuit Control Method of Induction Trash Can with Cold Cathode Ultraviolet Lamp Deodorization

(71) Applicant: Shi Ping Wang, Fuzhou, Fujian Province (CN)

(72) Inventors: Shi Ping Wang, Fuzhou (CN); Jiangqun Chen, Fuzhou (CN); Hao Jun Lin, Fuzhou (CN); Zhou Lin, Fuzhou (CN)

(73) Assignee: Fujian Nashida Electronic Incorporated Company, (omitted)

(21) Appl. No.: 18/204,945

(22) Filed: Jun. 2, 2023

(30) Foreign Application Priority Data
Apr. 14, 2022 (CN) 202210388196.0

(51) Int. Cl. A61L 2/10; A61L 2/24; A61L 9/01; B65F 1/16; B65F 7/00

(52) U.S. Cl. CPC A61L 2/10; A61L 2/24; A61L 9/01; B65F 1/1638; B65F 7/00; B65F 2210/168

(57) ABSTRACT

The present invention relates to a control technology of an electronic household trash container, in particular, a circuit control method of induction trash can with cold cathode ultraviolet lamp deodorization, which includes steps of setting a working cycle and a turn-off cycle of a ultraviolet sterilization and deodorization circuit, setting up an infrared pulse testing circuit to work for a predetermine number of times per second during the working cycle, wherein each working time of the infrared pulse testing circuit is 1 ms˜8 ms. The working time of the infrared pulse testing circuit is embedded in the working time of the sterilization and deodorization circuit so as to effectively implement the separation and integration of the working times thereof to effectively avoid the interference of the infrared pulse testing circuit, ensure the reliability of the induction of the lid, effectively prolong the service life of the cold cathode ultraviolet lamp, and achieve a more ideal sterilization and deodorization effect, with good safety performance, simple and reliable circuit structure, and low cost.

### BACKGROUND OF THE PRESENT INVENTION

### Field of Invention

The present invention relates to a control technology of an electronic household trash container, and more particularly to a circuit control method of induction trash with cold cathode ultraviolet lamp deodorization, which is adapted for the control of infrared induction circuit that is capable of sterilizing and deodorizing simultaneously by means of ultraviolet sterilization and ozone sterilization with cold cathode dual-band ultraviolet lamp.

### Description of Related Arts

Against the backdrop of the new coronavirus ravaging the world, non-contact sensor trash containers with sterilization and deodorization are favored by consumers. Most of the conventional sterilization and deodorization trash containers adopt the ozone method or the ultraviolet irradiation method. However, only a single method is used to sterilize and deodorize the effect is not effective enough. A dual-band cold cathode ultraviolet lamp can be used in the trash container to produce 254 nm wavelength and 185 nm wavelength ultraviolet light waves at the same time, wherein the 254 nm wavelength has a higher photon energy for irradiating microorganisms for sterilization, and the 185 nm wavelength light wave irradiates the air to convert O2 (oxygen) into O3 (ozone) in the air for ozone sterilization. The dispersion of ozone can just make up for the shortcomings of ultraviolet rays that only propagate along a straight line and is disinfection with dead angles, so that it is more practical and effective to apply both means. In addition, a dual-band cold cathode ultraviolet lamp utilized in the present invention can provide ultraviolet sterilization and ozone sterilization at the same time, so that the circuit structure and mechanical structure of the present invention are also simplified while its cost is effectively reduced.

Due to the high driving voltage of the cold cathode ultraviolet lamp, it needs to be equipped with a boost driving circuit, wherein the boost driving circuit has high-voltage pulses and harmonics. Because the induction trash can should have the function of being induced to open the lid at any time to meet the needs of users, the infrared pulse testing circuit is required to continuously emit infrared detection pulses, and the sterilization and deodorization circuit of the cold cathode ultraviolet lamp needs to work continuously for a period of time to achieve better results. Therefore, when the cold cathode ultraviolet lamp is installed in the induction trash can, its high-voltage pulse and harmonic will have a strong interference with the infrared pulse testing circuit with weak signal processing in the induction circuit, and the infrared pulse testing circuit works unreliable during the time period of common operation, resulting in the induction trash can cannot be inductively opened at any time, that significantly affects the use demand thereof.

In addition, most of the conventional ultraviolet lamps are in the working state without stopping when the lid is closed and standby. On the one hand, it can greatly affect the service life of the cold cathode ultraviolet lamp and increase the cost of utilization. Also, the excessive ozone generated by long-term work will diffuse and affect the environment, resulting in safety and health problems.

### SUMMARY OF THE PRESENT INVENTION

An object of the present invention is to provide a circuit control method of induction trash can with cold cathode ultraviolet lamp deodorization configured for avoiding signal interference, ensuring the reliability of inductive opening of the lid, prolonging the service life of ultraviolet lamp, and ensuring environmental safety in accordance with the shortcomings of the prior art.

The object of the present invention is implemented by:
a circuit control method of induction trash can with cold cathode ultraviolet lamp deodorization provides an infrared pulse testing circuit, a ultraviolet sterilization and deodorization circuit based on a cold cathode ultraviolet lamp, and a microcomputer controller and can lid driving circuit, wherein the infrared pulse testing circuit, the sterilization and deodorization circuit and the can lid driving circuit are all connected with a microcomputer controller;
including the following control steps:
S1: setting a working cycle and a turn-off cycle of the ultraviolet sterilization and deodorization circuit, wherein the working cycle is greater than 9 minutes, and setting the infrared pulse testing circuit to work n times per second in the working cycle of the ultraviolet sterilization and deodorization circuit, wherein each the working time of the infrared pulse testing circuit is 1 ms—8 ms, wherein a time between two adjacent operations of the infrared pulse testing circuit is a time interval;
S2: when the lid is closed and standby, entering a working state by the ultraviolet sterilization and deodorization circuit, and starting to time the working cycle;
S3: after the time interval, sending a trigger signal from the microcomputer controller to make the ultraviolet sterilization and deodorization circuit in a temporary turn-off state, and, during the temporary turn-off state, triggering the infrared pulse testing circuit by the microcomputer controller to start working and emitting infrared detection pulses by the infrared pulse testing circuit;
S4: when the infrared pulse testing circuit is working, if there is no object approaching the induction trash can, turning off the infrared pulse testing circuit after the working time of the infrared pulse testing circuit is ended, and then controlling the ultraviolet sterilization and deodorization circuit to start by the microcomputer controller, wherein the step S3 is returned until the working cycle of the ultraviolet sterilization and deodorization circuit is ended; and
S5: turning off the ultraviolet sterilization and deodorization circuit, turning off the cycle start timing, and returning to the step S2 after the turn-off cycle ends.

Accordingly, the infrared pulse testing circuit emits infrared detection pulses during its working time, and the ultraviolet sterilization and deodorization circuit is temporarily closed for 1 ms˜8 ms time. Taking an one-second cycle as an example, the infrared pulse testing circuit emits infrared detection pulses 3 times in each one-second cycle, and the ultraviolet sterilization and deodorization circuit is temporarily closed for a time of 1 ms each time, in which a working time ratio of the ultraviolet sterilization and deodorization circuit is 99.7% through computation; if the infrared pulse testing circuit emits infrared detection pulses 8 times in each one-second cycle and the ultraviolet sterilization and deodorization circuit is temporarily turned off for 8 ms, the working time ratio of the ultraviolet sterilization and deodorization circuit is 93.6% through computation. Such high working time ratio is equivalent to that the sterilization and deodorization circuit of the cold cathode ultraviolet lamp is almost in a continuous working state, which basically does not affect a continuity of ultraviolet irradiation and a change of ozone concentration.

The working time of the present invention based on the infrared pulse testing circuit is much smaller than the working time of the cold cathode ultraviolet lamp sterilization and deodorization circuit, while the ultraviolet sterilization and deodorization circuit adopts a predetermined number of temporary turn-off for extremely short time, such that the two working times thereof are being embedded control, that is embedding the working time of the infrared pulse testing circuit in the working cycle of the ultraviolet lamp sterilization and deodorization circuit, so as to effectively implement the separation and integration of time during the working state of the ultraviolet sterilization and deodorization circuit and the infrared pulse testing circuit. Therefore, on one hand, the ultraviolet sterilization and deodorization circuit will not interfere with the infrared pulse testing circuit to ensure the working stability of infrared pulse testing circuit and the reliability of the induction opening of the lid; on the other hand, the ultraviolet sterilization and deodorization circuit is basically maintained in a continuous working state during working hours that ensures the continuity of ultraviolet irradiation, so that the ultraviolet sterilization time and the accumulation requirements of ozone concentration can be satisfied. In addition, the ultraviolet sterilization and deodorization circuit is set with a turn-off cycle, which can effectively extend the service life of the ultraviolet lamp and can effectively control the ozone concentration in the trash container.

The present invention may be further specified as:

The number of infrared detection pulses emitted by the infrared pulse testing circuit in the working cycle of the ultraviolet sterilization and deodorization circuit is 3˜8 times per second, and the working time ratio of the ultraviolet sterilization and deodorization circuit is greater than 93.6%.

Regarding the ensuring of the infrared induction opening of the lid, the working time of the ultraviolet sterilization and deodorization circuit is ensured to have a high working time ratio, so as to ensure the continuity of ultraviolet irradiation and the accumulation of ozone concentration.

During the ultraviolet sterilization and deodorization circuit is in its working cycle, when the infrared pulse testing circuit works, such as an approaching object is detected through the emitted infrared detection pulse(s), the infrared detection pulse(s) is reflected by a surface of the object, and then the infrared pulse testing circuit processes the reflected signal from the object and feeding back to a microcomputer controller. The microcomputer controller controls the ultraviolet sterilization and deodorization circuit being maintained in the turn-off state, such that the working cycle temporarily stops timing, the microcomputer controller further triggers the can lid driving circuit, and the can lid is opened under the control of the can lid driving circuit. After the can lid is closed, the working cycle continues to time.

Because it only takes a few seconds from the induction trigger to open the lid of the induction trash can to close the can lid after disposal of the trash in the induction trash can, the ultraviolet sterilization and deodorization circuit returns to its original working state after closing the lid and continues the accumulation of the working cycle, that can not only ensure the integrity of the working cycle, but also basically will not affect the overall sterilization and deodorization effect while ensuring the induction opening of the lid being convenient to use.

During the ultraviolet sterilization deodorization circuit is in a turn-off state of the turn-off cycle, if an approaching object is detected through the emitted infrared detection pulse(s), the infrared detection pulse(s) is reflected by a surface of the object, and then the infrared pulse testing circuit processes the reflected signal from the object and feeding back to a microcomputer controller. Then, the turn-off cycle temporarily stops timing and the microcomputer controller further triggers the can lid driving circuit to open the can lid under a control by the can lid driving circuit. After the can lid is closed, the turn-off cycle in the microcomputer controller continues timing.

Likewise, the short opening and closing of the lid will not affect the integrity of the turn-off cycle. On the other hand, because ozone is an unstable gas, having a semi-decay phase, a fixed length of the turn-off cycle can be timely started after ozone is consumed to a certain extent to ensure the sterilization and deodorization effect.

The working cycle is 10˜30 minutes, and the turn-off cycle is 20˜60 minutes.

In order to ensure the sterilization and deodorization effect, the present invention takes a predetermined amount of time for ultraviolet irradiation to kill bacteria, and the concentration of ozone also needs a predetermined amount of time to accumulate, so that the ultraviolet sterilization and deodorization circuit is preferred to continue to work for more than 10 minutes. Considering the rate of bacterial growth and ozone consumption, the turn-off cycle should be less than 60 minutes.

In view of above, the present invention provides an circuit control method of induction trash can with cold cathode ultraviolet lamp deodorization, which embeds the working time of the infrared pulse testing circuit into the working time of the ultraviolet lamp sterilization and deodorization circuit, so as to effectively implement the separation and integration of time during the working state of the ultraviolet sterilization and deodorization circuit and the infrared pulse testing circuit, avoid the interference of the sterilization and deodorization circuit of the cold cathode ultraviolet lamp with respect to the infrared test circuit, accomplish that the lid can be opened inductively at any time in the standby state, prolong the service life of the cold cathode ultraviolet lamp, control the ozone concentration in the trash container, and achieve a more ideal sterilization and deodorization effect simultaneously. The present invention has good safety performance, simple and reliable circuit structure, low cost, and strong practical application value.

### BRIEF DESCRIPTION OF THE DRAWINGS

FIG. 1 is a schematic diagram of a circuit control method of induction trash can with cold cathode ultraviolet lamp deodorization according to a preferred embodiment of the present invention.

FIG. 2 is a schematic diagram illustrating a working sequence of an infrared pulse testing circuit when an ultraviolet sterilization and deodorization circuit enters a working state of a working cycle of the circuit control method of induction trash can of deodorizing with cold cathode ultraviolet lamp deodorization according to the above preferred embodiment of the present invention.

FIG. 3 is a schematic diagram illustrating an intermittent working sequence of the ultraviolet sterilization and deodorization circuit during a standby state of closing the lid of the circuit control method of induction trash can of deodorizing with cold cathode ultraviolet lamp deodorization according to the above preferred embodiment of the present invention.

The reference characters of the accompanying drawings are specifically as follows: 1—reflective-type infrared pulse testing circuit, 11—infrared emitting tube, 12—infrared receiving tube, 2—microcomputer controller, 3—sterilization and deodorization circuit of cold cathode ultraviolet lamp, 31—boost driving circuit, 32—cold cathode ultraviolet lamp, 4—can lid driving circuit, 5—object.

The present invention is further described below in combination with the preferred embodiment.

### DETAILED DESCRIPTION OF THE PREFERRED EMBODIMENT

### Preferred Embodiment

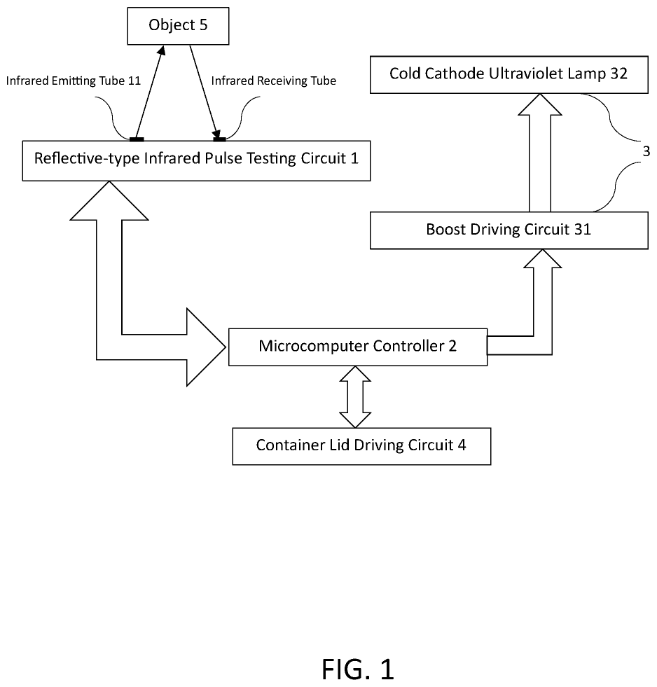

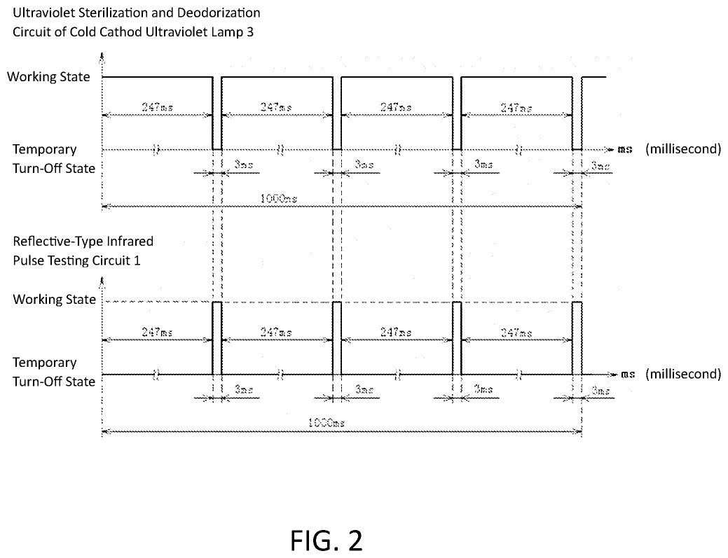

Referring to FIG. 1 and FIG. 2 of the drawings, a circuit control method of an induction trash can with a cold cathode ultraviolet lamp deodorization is illustrated, wherein a control circuit comprises an infrared pulse testing circuit 1, an microcomputer control 2, an ultraviolet sterilization and deodorization circuit of cold cathode ultraviolet lamp 3, and a can lid driving circuit 4. The infrared pulse testing circuit 1 is a reflective-type infrared pulse testing circuit, which comprises an infrared emitting tube 11 and an infrared receiving tube 12. The microcomputer control 2 controls a coordinated work of each circuit of the present invention. The ultraviolet sterilization and deodorization circuit of cold cathode ultraviolet lamp 3 consists of a boost driving circuit 31 and a dual-band cold cathode ultraviolet lamp 32, wherein the boost driving circuit 31 converts a safety low-voltage power supply into a power supply of about 370V for the dural-band cold cathode ultraviolet lamp 32 for use, and the can lid driving circuit 4 is configured to drive the opening and closing of the can lid. In order to prevent ultraviolet rays from injuring human eyes and skin, only when the trash container is in a lid closing standby state, the ultraviolet sterilization and deodorization circuit 3 is allowed to work. When the ultraviolet sterilization and deodorization circuit 3 works, the induction trash can should have the function of induction opening of the lid. Because the ultraviolet sterilization and deodorization circuit 3 has a relatively long continuous working time and the lid is not suitable to be induced to open. It may cause inconvenience to the use of induction trash can. During the standby state of closing the lid and the ultraviolet sterilization and deodorization circuit 3 is in the working state: when the reflective-type infrared pulse testing circuit 1 is required to work, the microcomputer controller 2 controls the ultraviolet sterilization and deodorization circuit 3 in a temporary closing state, and thus after the reflective-type infrared pulse testing circuit 1 completes the test, the reflective-type infrared pulse testing circuit 1 is turned off and the ultraviolet sterilization and deodorization circuit 3 is turned on immediately if there is no object 5 approaching an induction window of the induction trash can, such that the interference of ultraviolet sterilization and deodorization circuit 3 to the reflective-type infrared pulse testing circuit 1 is avoided, and that, at the same time, a working time of the reflective-type infrared pulse testing circuit 1 is much less than the working time of the sterilization and deodorization circuit cold cathode ultraviolet lamp 3. Because the sterilization and deodorization circuit of cold cathode ultraviolet lamp 3 is preferred to last for more than 10 minutes for an ideal sterilization effect. And, the ultraviolet irradiation requires a predetermined amount of time to kill the bacteria and the concentration of ozone requires a predetermined amount of time to accumulate. Accordingly, the relatively short temporary turn-off of the sterilization and deodorization circuit of cold cathode ultraviolet lamp 3 during its working state is almost equivalent to the sterilization and deodorization circuit of cold cathode ultraviolet lamp 3 is working continuously, that basically does not affect the continuity of ultraviolet radiation and the change of ozone concentration.

Referring to FIG. 2 of the drawings, when the lid is closed and the sterilization and deodorization circuit of cold cathode ultraviolet lamp 3 is in the working state, in each one-second cycle of the present embodiment, the reflective-type infrared pulse testing circuit 1 works 4 times, wherein the working time of each time is 3 ms and an infrared detection pulse is emitted by the infrared emitting tube 11 to an induction area. To each emission of the infrared detection pulse, the time of the temporary turn-off of the sterilization and deodorization circuit cold cathode ultraviolet lamp 3 is 3 ms, while an interval time is 247 ms. In each one-second cycle, the reflective-type infrared pulse testing circuit 1 emits an average of 4 infrared detection pulses, which can ensure the induction trash cannot respond slowly and not to take too long. That is 4 infrared detection pulses is emitted per second while the sterilization and deodorization circuit of cold cathode ultraviolet lamp 3 is temporarily closed for 3 ms each time, such that a working time ratio of the sterilization and deodorization circuit cold cathode ultraviolet lamp 3 is 98.8% through computation. Such high working time ratio is equivalent to that the sterilization and deodorization circuit of cold cathode ultraviolet lamp is almost continuously working and basically does not affect the continuity of ultraviolet radiation and the change of ozone concentration.

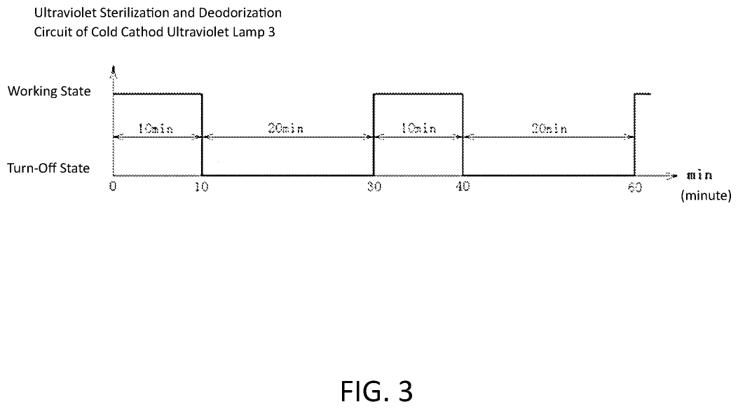

In the preferred embodiment, a U-shaped 3.3-watt dual-band cold cathode ultraviolet lamp 32 is embodied and its life span is about 13000 hours, wherein the working cycle of the dual-band cold cathode ultraviolet lamp 32 is 10 minutes, the turn-off cycle is 20 minutes. As shown in FIG. 3, it works repeatedly in an intermittence manner, such that calculated as 24 hours a day and the dual-band cold cathode ultraviolet lamp 32 actually works for up to 8 hours a day, the dual-band cold cathode ultraviolet lamp 32 can be used for more than 4 years, that basically meets the market requirement. Also, at the same time, that will not cause the ozone concentration in the trash container to be too high, wherein a small portion of the ozone will diffuse out when the lid is opened (a specific gravity of ozone is heavier than the air), so that the diffused ozone concentration will not exceed the safety and health standard of 1 ppm. While comprehensively considering that the time of most bacterial reproduction generation is 2030 minutes, as well as considering that ozone is an unstable gas and its semi-decay phase is only about 30 minutes, the dural-band cold cathode ultraviolet lamp 32 is preferred to continue to work for 10 minutes and stop working for 20 minutes according to the preferred embodiment.

During a standby state of closing the lid of the induction trash can, the sterilization and deodorization circuit of cold cathode ultraviolet lamp 3 may be in working state or turn-off state. When the object 5 is approaching an induction window of the induction trash can, the infrared detection pulse emitted by infrared emitting tube 11 is reflected by a surface of the object 5 and received by infrared receiving tube 12, wherein the weak received signal is then amplified by an amplification of reflective-type infrared pulse testing circuit 1 and fed back to microcomputer controller 2. Then, the microcomputer controller 2 controls the boost driving circuit 31 to stop working, thereby cutting off a high-voltage power supply of the dual-band cold cathode ultraviolet lamp 32, making the sterilization and deodorization circuit of cold cathode ultraviolet lamp 3 in the turn-off state, avoiding ultraviolet radiation hurting human eyes and skin, and then controlling the can lid to open. After the trash is disposed in the induction trash can, the can lid is closed and the sterilization and deodorization circuit of cold cathode ultraviolet lamp 3 returns to the original working state or closing state, wherein the working state time or the closing state time of the sterilization and deodorization circuit of cold cathode ultraviolet lamp 3 is computed as an accumulation time that lasts the previous cycle. Because only a few seconds is taken for the induction trash can to open the lid, from the induction trigger to open and to the closing of the lid after the trash is disposed in the induction trash can, the sterilization and deodorization circuit of cold cathode ultraviolet lamp 3 returns to its original state and continues the accumulation time with the previous cycle, that basically does not affect the overall sterilization and deodorization effect.

The part not described in the present invention can be the same as the prior art.

1. A circuit control method of induction trash can with cold cathode ultraviolet lamp deodorization, which comprises an infrared pulse testing circuit, an ultraviolet sterilization and deodorization circuit of cold cathode ultraviolet lamp, a microcomputer controller, and a can lid driving circuit, wherein the infrared pulse testing circuit, the sterilization and deodorization circuit and the can lid driving circuit are connected with the microcomputer controller, the circuit control method comprising steps of:
S1: setting a working cycle and turn-off cycle of the ultraviolet sterilization and deodorization circuit, wherein the working cycle is greater than 9 minutes, and setting the infrared pulse testing circuit to work a predetermined number of times per second during the working cycle of the ultraviolet sterilization and deodorization circuit, wherein each the working time of the infrared pulse testing circuit is 1 ms˜8 ms;
S2: when a lid of the induction trash can is closed and standby, entering a working state by the ultraviolet sterilization and deodorization circuit, and starting to time the working cycle;
S3: after an interval time, sending a trigger signal from the microcomputer controller to make the ultraviolet sterilization and deodorization circuit in a temporary turn-off state, and, during the temporary turn-off state, triggering the infrared pulse testing circuit by the microcomputer controller to start working and emitting infrared detection pulses by the infrared pulse testing circuit;
S4: when the infrared pulse testing circuit is working, if there is no object approaching the induction trash can, turning off the infrared pulse testing circuit after the working time of the infrared pulse testing circuit is ended, and then controlling the ultraviolet sterilization and deodorization circuit to start by the microcomputer controller, wherein the step S3 is returned until the working cycle of the ultraviolet sterilization and deodorization circuit is ended; and
S5: turning off the ultraviolet sterilization and deodorization circuit, starting to time the turn-off cycle, and returning to the step S2 after the turn-off cycle ends.

2. The circuit control method according to claim 1, wherein the infrared pulse testing circuit emits the infrared detection pulses for 3˜8 times in each second in the working cycle of the ultraviolet sterilization and deodorization circuit, and a working cycle ratio of the ultraviolet sterilization and deodorization circuit is greater than 93.6%.

3. The circuit control method according to claim 1, wherein, during the working cycle of the ultraviolet sterilization and deodorization circuit, when the infrared pulse testing circuit works, such that the approaching of the object is induced through the emitted infrared detection pulses reflected by a surface of the object, wherein the infrared pulse testing circuit processes signals reflected by the object and feeds back to the microcomputer controller, wherein while the ultraviolet sterilization and deodorization circuit is controlled by the microcomputer controller is in the turn-off state, the working cycle temporarily stops to time and the microcomputer controller further triggers the can lid driving circuit which controls the lid to open, wherein after the lid is closed, the working cycle continues to be timed.

4. The circuit control method according to claim 1, wherein when the ultraviolet sterilization and deodorization circuit is in the turn-off state during the turn-off cycle, if the emitted infrared detection pulses induce the approaching of the object that the infrared detection pulse are reflected by the surface of the object, the infrared pulse testing circuit processes the signal reflected by the object and feeds back to the microcomputer controller, and then the turn-off cycle temporarily stops to time, wherein the microcomputer controller further triggers the can lid driving circuit, such that the lid is controlled by the can lid driving circuit to open, wherein after lid is closed, the turn-off cycle in the microcomputer controller continues timing.

5. The circuit control method according to claim 1, wherein the working cycle is 10˜30 minutes and the turn-off cycle is 20˜60 minutes.

6. A circuit control method of an induction trash can with cold cathode ultraviolet lamp deodorization, comprising steps of:
S1: setting a working cycle and turn-off cycle of ultraviolet sterilization and deodorization circuit, wherein the working cycle is greater than 9 minutes, and setting an infrared pulse testing circuit to work a predetermined number of times per second during the working cycle of the ultraviolet sterilization and deodorization circuit, wherein each the working time of the infrared pulse testing circuit is 1 ms˜8 ms;
S2: when a lid of the induction trash can is closed and standby, entering a working state by the ultraviolet sterilization and deodorization circuit, and starting to time the working cycle;
S3: after an interval time, sending a trigger signal from a microcomputer controller to make the ultraviolet sterilization and deodorization circuit in a temporary turn-off state, and, during the temporary turn-off state, triggering the infrared pulse testing circuit by the microcomputer controller to start working and emitting infrared detection pulses by the infrared pulse testing circuit;
S4: when the infrared pulse testing circuit is working, if there is no object approaching the induction trash can, turning off the infrared pulse testing circuit after the working time of the infrared pulse testing circuit is ended, and then controlling the ultraviolet sterilization and deodorization circuit to start by the microcomputer controller, wherein the step S3 is returned until the working cycle of the ultraviolet sterilization and deodorization circuit is ended; and
S5: turning off the ultraviolet sterilization and deodorization circuit, turning off the cycle start timing, and returning to the step S2 after the turn-off cycle timer ends.

7. The circuit control method according to claim 1, wherein the infrared pulse testing circuit emits the infrared detection pulses for 3˜8 times in each second in the working cycle of the ultraviolet sterilization and deodorization circuit, and a working cycle ratio of the ultraviolet sterilization and deodorization circuit is greater than 93.6%.

8. The circuit control method according to claim 6, wherein, during the working cycle of the ultraviolet sterilization and deodorization circuit, when the infrared pulse testing circuit works, such that the approaching of the object is induced through the emitted infrared detection pulses reflected by a surface of the object, wherein the infrared pulse testing circuit processes signals reflected by the object and feeds back to the microcomputer controller, wherein while the ultraviolet sterilization and deodorization circuit is controlled by the microcomputer controller is in the turn-off state, the working cycle temporarily stops to time and the microcomputer controller further triggers the can lid driving circuit which controls the lid to open, wherein after the lid is closed, the working cycle continues to be timed.

9. The circuit control method according to claim 7, wherein, during the working cycle of the ultraviolet sterilization and deodorization circuit, when the infrared pulse testing circuit works, such that the approaching of the object is induced through the emitted infrared detection pulses reflected by a surface of the object, wherein the infrared pulse testing circuit processes signals reflected by the object and feeds back to the microcomputer controller, wherein while the ultraviolet sterilization and deodorization circuit is controlled by the microcomputer controller is in the turn-off state, the working cycle temporarily stops to time and the microcomputer controller further triggers the can lid driving circuit which controls the lid to open, wherein after the lid is closed, the working cycle continues to be timed.

10. The circuit control method according to claim 6, wherein when the ultraviolet sterilization and deodorization circuit is in the turn-off state during the turn-off cycle, if the emitted infrared detection pulses induce the approaching of the object that the infrared detection pulse are reflected by the surface of the object, the infrared pulse testing circuit processes the signal reflected by the object and feeds back to the microcomputer controller, and then the turn-off cycle temporarily stops to time, wherein the microcomputer controller further triggers the can lid driving circuit, such that the lid is controlled by the can lid driving circuit to open, wherein after lid is closed, the turn-off cycle in the microcomputer controller continues timing.

11. The circuit control method according to claim 7, wherein when the ultraviolet sterilization and deodorization circuit is in the turn-off state during the turn-off cycle, if the emitted infrared detection pulses induce the approaching of the object that the infrared detection pulse are reflected by the surface of the object, the infrared pulse testing circuit processes the signal reflected by the object and feeds back to the microcomputer controller, and then the turn-off cycle temporarily stops to time, wherein the microcomputer controller further triggers the can lid driving circuit, such that the lid is controlled by the can lid driving circuit to open, wherein after lid is closed, the turn-off cycle in the microcomputer controller continues timing.

12. The circuit control method according to claim 8, wherein when the ultraviolet sterilization and deodorization circuit is in the turn-off state during the turn-off cycle, if the emitted infrared detection pulses induce the approaching of the object that the infrared detection pulse are reflected by the surface of the object, the infrared pulse testing circuit processes the signal reflected by the object and feeds back to the microcomputer controller, and then the turn-off cycle temporarily stops to time, wherein the microcomputer controller further triggers the can lid driving circuit, such that the lid is controlled by the can lid driving circuit to open, wherein after lid is closed, the turn-off cycle in the microcomputer controller continues timing.

13. The circuit control method according to claim 9, wherein when the ultraviolet sterilization and deodorization circuit is in the turn-off state during the turn-off cycle, if the emitted infrared detection pulses induce the approaching of the object that the infrared detection pulse are reflected by the surface of the object, the infrared pulse testing circuit processes the signal reflected by the object and feeds back to the microcomputer controller, and then the turn-off cycle temporarily stops to time, wherein the microcomputer controller further triggers the can lid driving circuit, such that the lid is controlled by the can lid driving circuit to open, wherein after lid is closed, the turn-off cycle in the microcomputer controller continues timing.

14. The circuit control method according to claim 6, wherein the working cycle is 10˜30 minutes and the turn-off cycle is 20˜60 minutes.

15. The circuit control method according to claim 7, wherein the working cycle is 10˜30 minutes and the turn-off cycle is 20˜60 minutes.

16. The circuit control method according to claim 8, wherein the working cycle is 10˜30 minutes and the turn-off cycle is 20˜60 minutes.

17. The circuit control method according to claim 9, wherein the working cycle is 10˜30 minutes and the turn-off cycle is 20˜60 minutes.

18. The circuit control method according to claim 11, wherein the working cycle is 10˜30 minutes and the turn-off cycle is 20˜60 minutes.

19. The circuit control method according to claim 12, wherein the working cycle is 10˜30 minutes and the turn-off cycle is 20˜60 minutes.

20. The circuit control method according to claim 13, wherein the working cycle is 10˜30 minutes and the turn-off cycle is 20˜60 minutes.
